(19) United States
(12) Patent Application Publication
(10) Pub. No.: US 2014/0328447 A1
(43) Pub. Date: Nov. 6, 2014

(54) SYSTEMS AND METHODS FOR COMPUTED TOMOGRAPHY (CT) IMAGING USING VARIABLE IMAGE QUALITY FACTORS OR IMAGE CAPTURE SETTINGS IN A SINGLE ACQUISITION

(71) Applicant: Duke University, Durham, NC (US)

(72) Inventor: Lynne M. Hurwitz Koweek, Durham, NC (US)

(21) Appl. No.: 14/267,868

(22) Filed: May 1, 2014

Related U.S. Application Data
(60) Provisional application No. 61/817,960, filed on May 1, 2013.

(51) Int. Cl. A61B 6/00; A61B 6/03

(52) U.S. Cl. CPC A61B 6/405; A61B 6/032
USPC 378/4

(57) ABSTRACT

Systems and methods for computed tomography (CT) imaging using variable image quality factors or image capture settings in a single acquisition are disclosed. According to an aspect, a method includes using a CT system to capture CT image data of an object in a single acquisition. The method also includes adjusting one or more image quality factor or image capture settings of the CT system during capture of the CT image data of the object. Further, the method includes processing the CT image data for presentation based on the one or more image quality factor or image capture settings.

### CROSS REFERENCE TO RELATED APPLICATION

This application claims the benefit of U.S. Provisional Patent Application No. 61/817,960, filed May 1, 2013 and titled SYSTEMS AND METHODS FOR THE OPTIMIZATION OF MULTI-DETECTOR COMPUTED TOMOGRAPHIC WHOLE BODY IMAGING, the disclosure of which is incorporated herein by reference in its entirety

### TECHNICAL FIELD

The presently disclosed subject matter relates to imaging. Particularly, the presently disclosed subject matter relates to systems and method for computed tomography (CT) imaging using different image capture settings.

### BACKGROUND

CT is a technology utilizes computer-processed X-rays to produce tomographic images (virtual “slices”) of specific areas of a scanned object. Digital geometry processing may be used to generate a three-dimensional image of the inside of an object from a large series of two-dimensional radiographic images taken around a single axis of rotation. Medical imaging is an important application of CT technology.

Advances in CT technology are related in great part to speed of image acquisition. Particularly, faster rotation time and increased number of detectors allow for larger z-axis coverage during a single acquisition, resulting in the ability to acquire full body coverage as a single continuous dataset. The benefits of a single data acquisition include lower total volume of contrast administration and fewer misregistration artifacts as compared to acquisition of the same data using multiple sequential acquisitions. Additionally, a single acquisition technique can allow for a comprehensive road map of vascular anatomy devoid of variations in contrast opacification encountered with sequential acquisitions. Imaging over an extended z-axis region with a single continuous acquisition as compared to sequential acquisitions can reduce radiation dose due to the removal of repeat imaging at the same z-axis location that otherwise occur when two adjacent areas are imaged independently to assure complete anatomic coverage. The current limitation with a single continuous acquisition technique is selection of one set of parameters for use along the entire z-axis which may not be specific and/or optimized to the different anatomic regions that are being scanned.

Automatic tube current modulation allows for reduction in radiation dose while maintaining constant image quality based on body habitus and beam attenuation in different anatomic regions as determined by scout images and real time adjustment for modulation in the x-axis, y-axis, and z-axis. However, utilization of this technology requires the user to input an appropriate noise level or image quality factor that will balance image quality and radiation dose. The choice of noise level/image quality factor can vary depending on the indication of the study, in addition to the pathology and anatomic region being imaged. Currently, there is no flexibility to change these parameters for a single acquisition over different anatomic regions during a single image acquisition. Presently, combined chest and abdomen/pelvis imaging can be accomplished in one of two manners. In one approach, two separate scan acquisitions are utilized, each with their own noise index/image quality factor, contrast administration, and redundant imaging over the caudal portions of the chest and cranial portions of the abdomen to insure adequate inclusion of the entire z-axis. In a second approach, a single acquisition is performed over the desired z-axis coverage using one specific noise level/image quality factor and a single administration of contrast for both anatomic regions of interest. The limitation with this second approach to imaging is the selection of a single image quality factor that is appropriate for both the chest and abdomen/pelvis with currently available software.

In view of the foregoing, there is a need for improved CT imaging systems and techniques.

### BRIEF SUMMARY

Disclosed herein are systems and methods for CT imaging using different image capture settings. According to an aspect, a method includes using a CT system to capture CT image data of an object in a single acquisition. The method also includes adjusting one or more image capture settings of the CT system during capture of the CT image data of the object. Further, the method includes processing the CT image data for presentation based on the one or more image capture settings.

### BRIEF DESCRIPTION OF THE SEVERAL VIEWS OF THE DRAWINGS

The foregoing aspects and other features of the present subject matter are explained in the following description, taken in connection with the accompanying drawings, wherein:

FIG. 1 is a perspective view of an example CT system 100 in accordance with embodiments of the present subject matter;

FIG. 2 is a flow chart of an example method for CT imaging using different image capture settings;

FIG. 3 is a scout image of a phantom with superimposed cartographic depiction of the three protocols used and the respective noise indices in relationship to anatomic position; prototype ViSIoN (dotted line), Sequential (dashed line), and C&AP (solid line) acquisitions are represented (B);

FIG. 4 is a graph showing measured organ dose values for the three protocols (C& AP Sequential, Single C&AP, prototype ViSIoN C&AP) using Auto MA settings (*denotes recalibrated dose values);

FIG. 5 is a graph showing measured organ dose values for the three protocols (C& AP Sequential, Single C&AP, prototype ViSIoN C&AP) using Smart MA settings (*denotes recalibrated dose values);

FIGS. 6A and 6B are axial images of the lower thorax (FIG. 6A) and upper abdomen (FIG. 6B) showing ROI locations (circles) used for noise measurement in the central mediastinum (black circle), central left lung (white circle), midline anterior abdominal wall (circle) central abdomen (circle);

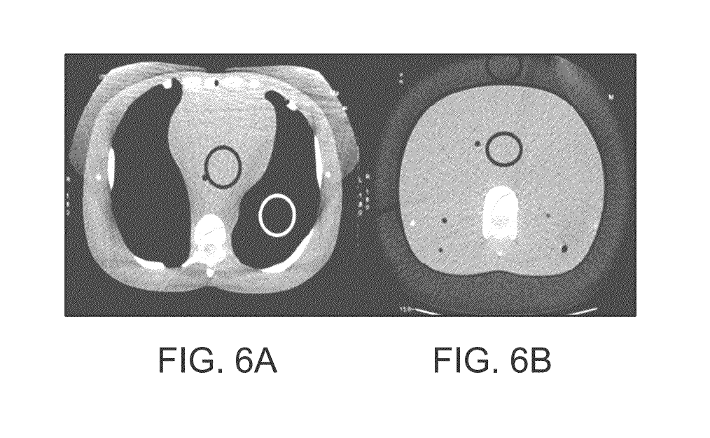

FIG. 7 shows radiochromic films and graphs showing z-axis radiation dose distribution for prototype ViSIoN software incorporating point of change in NI for both Smart mA (top) and Auto mA (bottom), (arrow denotes point of NI change); and

FIGS. 8A and 8B show an image localizer image (FIG. 8A) and tube current as a function of Z-Axis location (FIG. 8B) for both Smart mA and Auto mA settings for each CT protocol.

### DETAILED DESCRIPTION

For the purposes of promoting an understanding of the principles of the present disclosure, reference will now be made to various embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (ie. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

In accordance with embodiments, systems and methods disclosed herein provide for acquisition of one dataset over an extended z-axis range (ie., two or more different anatomic regions) along with the ability of the user to adjust (or select) anatomic specific CT parameters (e.g., mA, kVp, FOV, rotation time, and the like). The result being a single data acquisition obtained using anatomic region specific image quality parameters. The initial radiographic scout film is used to delineate each specific anatomic region to be imaged and subsequent image acquisition utilizes individually prescribed specific image quality factor(s) during image acquisition for each uniquely identified portion of the entire z-axis coverage (e.g., chest, abdomen, neck, etc.). Example advantages of this technique include improved speed and reduced radiation dose when scanning over an extended z-axis scanning across multiple anatomic regions. Further, the systems and methods disclosed herein can provide an alternate to sequential acquisitions with anatomic specific image quality settings. In an example, systems and methods disclosed herein can provide for whole body MDCT imaging that allows for anatomic specific image quality factors to be chosen for the chest and abdomen/pelvis during a single acquisition and assess the change in radiation dose, total scan acquisition time, and image quality.

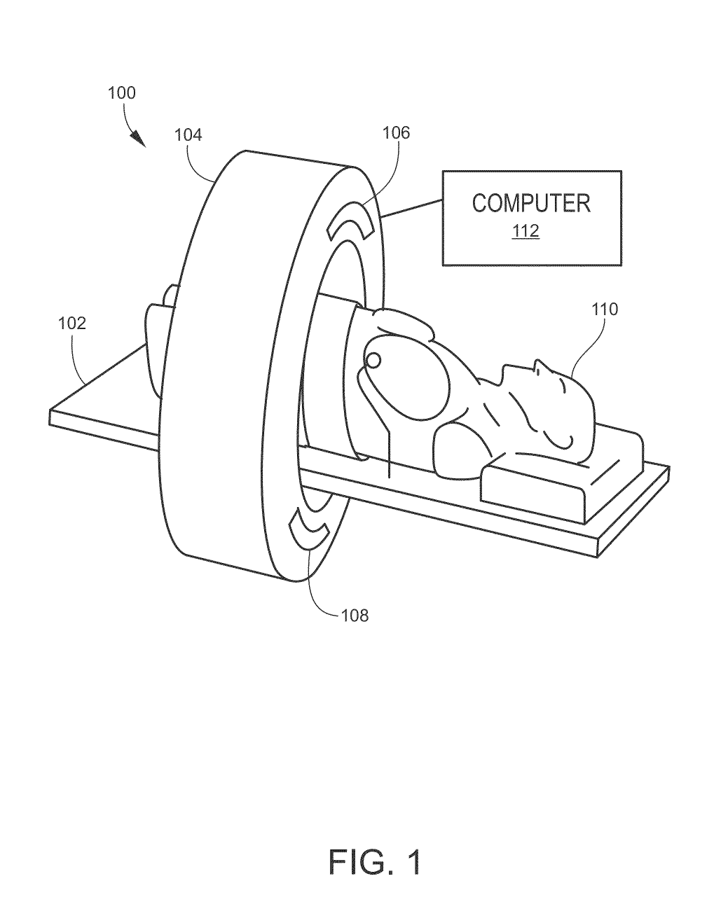

FIG. 1 illustrates a perspective view of an example CT system 100 in accordance with embodiments of the present subject matter. Referring to FIG. 1, the system 100 includes a subject-receiving area in the form of a moveable bed or table 102 and an enclosure 104 partially surrounding the bed 102. The enclosure 104 can include a low radiation source, such as an X-ray source 106, and a detector assembly 108. The X-ray source 106 is configured to generate X-rays and direct the X-rays towards an object on the bed 102. The X-ray detector assembly 108 may detect the X-rays after interaction of the X-rays with the object.

The enclosure 104 may be a cylindrical in shape and positioned about the bed 102 defining a tunnel therein. The X-ray source 106 may be, for example, an X-ray tube mounted in the enclosure 104 so as to project a low energy radiation beam towards the radiation detector assembly 108 after passage through an object 110.

The detector assembly 108 may be located in the enclosure opposite the X-ray source 106 and therefore beyond the bed 102. The object 110 may include, but is not limited to, a human, an animal, or an imaging phantom. In this example, the object 110 is an adult anthropomorphic phantom with supine breast attachments for use in measuring absorbed radiation dose to various organs.

The system 100 or another suitable system may be utilized for implementing the methods disclosed herein. In accordance with embodiments, systems and methods are disclosed for assessing radiation dose distribution, image quality, and scan time for a single acquisition of the chest, abdomen and pelvis multi-detector computer tomography (MDCT) acquisition using anatomical specific image quality setting thereby providing for the optimization of multi-detector computed tomographic whole body imaging. For example, the system 100 may be configured for MDCT acquisition.

A computer 112 may be operatively configured to implement CT imaging within the CT system 100. The computer 112 may include hardware, software, firmware, or combinations thereof for implementing the functions disclosed herein. For example, the computer 112 may include one or more processors and memory. Further, the computer may include a suitable user interface, such as a display, for presenting CT images.

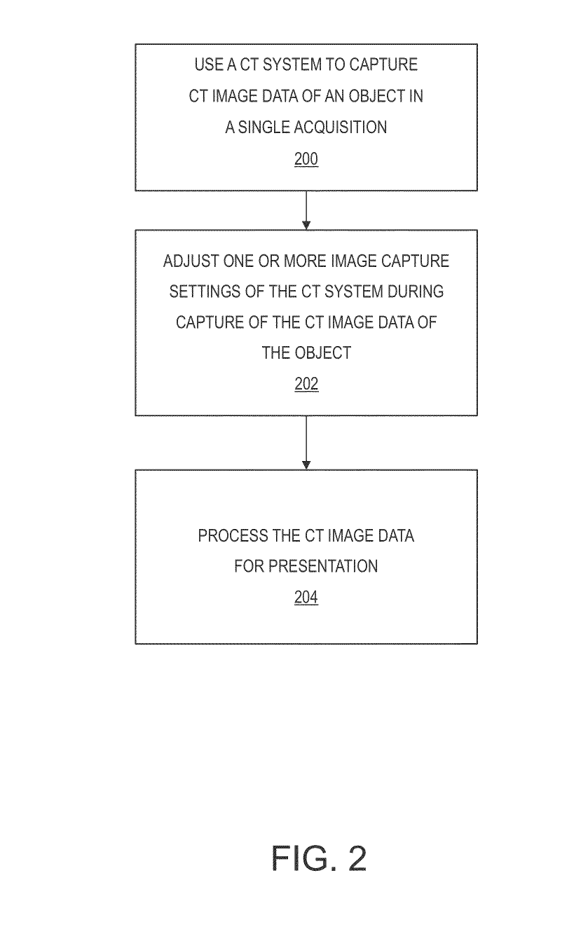

In accordance with embodiments of the present subject matter, FIG. 2 illustrates a flow chart of an example method for CT imaging using different image capture settings. In this example, the method is described as being implemented by the CT system 100 shown in FIG. 1, although it should be understood that the method may be implemented by any other suitable type of imaging system.

Referring to FIG. 2, the method includes using a CT system to capture CT image data of a single object in a single acquisition. For example, the CT system 100 shown in FIG. 1 can capture CT image data of the object 110 in a single acquisition. The table 102 may be suitably moved along a z-axis while CT image data is acquired. In an example, a first set of CT image data may be captured with the CT system set to one image capture setting. Example image capture settings includes, but are not limited to, mA, kVp, FOV, rotation time, pitch, and the like. The first set of CT image data may be captured of a particular portion of the object 110. For example, the first set of CT image data may be captured of an upper portion of a human. Subsequently, a second set of CT image data may be captured of another portion of the object 110. For example, the second set of CT image data may be captured of a lower portion of the human.

The method of FIG. 2 includes adjusting one or more image capture settings of the CT system during capture of the CT image data of the object. Continuing the aforementioned example, the image capture settings can be adjusted to different settings as the CT image data of the object 110 is captured. Further, for example, one or more image quality factors of the CT system 100 can be adjusted during capture of the CT image of the object. The image quality factors may be adjusted as the CT system 100 acquires images of different portions of the human anatomy during a single acquisition. In other words, the image quality factors set for the CT system 100 may be different in a single acquisition while different anatomical features of a human body are imaged.

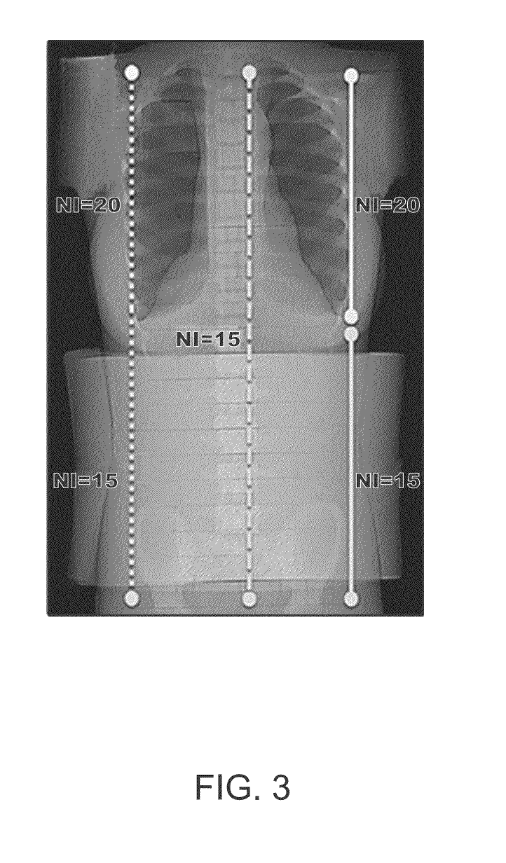

The method of FIG. 3 includes processing the CT image data for presentation based on the one or more image capture settings. Continuing the aforementioned example, the captured CT image data may be communicated to the computer 112 and stored therein. The computer 112 may subsequently process the CT image data based on the different image capture settings. Further, the computer 112 may present (e.g., display) the CT images.

In accordance with embodiments of the present subject matter, processed image CT data may be assessed based on image quality. Further, imaging techniques disclosed herein may be assessed based on a determined radiation dosage level at different portions of an object subsequent to use of the CT system as disclosed herein. In an example, absorbed organ doses are measured using thirteen (13) MOSFET detectors on a 64-MDCT scanner (e.g., a Discovery CT,750 HD, GE Healthcare) with three protocols: Chest and Abdomen/Pelvis dual noise level sequential acquisition with Noise Index (NI) of 20 and 15, respectively (minimal delay between regions); Chest and Abdomen/Pelvis single noise level acquisition with a NI of 15 (no time delay between regions); and Chest and Abdomen/Pelvis prototype Variable Setting Image Noise (ViSIoN) software with dual noise level single acquisition with NI of 20 and 15, respectively (no time delay between anatomic regions). Auto mA and Smart mA may be utilized for each of the three protocols for a total of six independent scans. In experiments, total scan time, CTDI and DLP was recorded for each protocol. Noise was measured to evaluate image quality. Radiation dose distribution was measured with radiochromic film analysis.

In practice, the prototype ViSIoN protocol, on average, demonstrated the greatest decrease in absorbed organ dose compared to the single NI protocol with greatest organ dose reduction to the liver (43%), lung (36%), kidney (36%), breast (33%). Total DLP was reduced using the prototype ViSIoN protocol, 11% compared to the dual noise level sequential protocol and 18% compared to the single noise level acquisition protocoL Scan time was decreased for the prototype ViSIoN protocol versus dual noise level sequential acquisition MDCT protocol (3.48 seconds). Image quality was maintained between protocols.

MDCT scanning over multiple anatomic regions can be achieved with dedicated region specific image quality factors for the chest and abdomen/pelvis in a single acquisition with a reduction in radiation dose and scan time as compared to single acquisition noise index (NI) chest and abdomen/pelvis and a dual NI sequential chest and abdomen/pelvic MDCT acquisition.

In a study, an adult anthropomorphic phantom (model 701-D; CIRS, Norfolk, Va.) with supine breast attachments (model 701-BR-02; CIRS, Norfolk, Va.) was used to measure absorbed radiation dose to various organs. The phantom used in this study was previously validated for human organ dosimetry measurements. The specific dimensions of this example phantom are as follows: weight, 73 kg; height, 173 cm; and chest, 23×32 cm. The phantom includes bone, lung, brain, spinal cord, and soft-tissue compositions. For example, the object 110 may be a phantom used for this assessment. The phantom may be subdivided into 39 contiguous 2.5-cm thick sections. Each section contains several 5-mm-diameter holes through which detectors are placed for organ dose measurements. The phantom has assignable anatomic locations, and each hole location was optimized for precise dosimetry within a specific internal organ that can be referenced to the manufacturer's user manual. Tissue-equivalent plugs may fill the holes when they are not being used. The supine breasts represent the clinically relevant shape of the breast in the supping position and are attachable to the main body. The supine breasts are made of a 50:50 glandular-to-adipose formula. Specific holes are located in the breasts 1 cm below the skin surface for detector placement. One fat layer was placed around the abdomen to simulate an average adult patient body habitus.

To determine organ dose, thirteen metal oxide semiconductor field effect transistor (MOSFET) dosimeters (model 1002RD, Best Medical) with active detector areas of 200×200 μm (total dimensions, 2.5 mm width×1.3 mm thickness×8 mm length) were placed in defined anatomic locations in the chest, abdomen, and pelvis. Each detector was calibrated in air using a conventional x-ray tube with added filtration that simulated the beam quality of the 120 kVp CT beam used in this experiment (HVL=7.26 mm Al); individual calibration factors for all detectors were calculated and archived. Any suitable calibration method and validation of MOSFET methods may be utilized. The lower limit of detection of absorbed dose for the MOSFETs with AutoSense system was 1.50 mGy.

MOSFET detectors were inserted into the predrilled holes of the anthropomorphic phantom and distributed into the following locations: bone marrow in the thoracic and lumbar spine and pelvis, thyroid, thymus, mid and lower locations of the lung, esophagus, kidney, small intestine, large intestine, uterus, bladder, and the breast, left and right. The MOSFET reader was connected to a laptop computer, and the data were read immediately after each CT scan. The software (model TN-RD-60; and model TN-RD-70-W, Best Medical, Canada) stored acquired data in centigrays (cGy). The phantom was scanned three times per protocol and the organ doses were averaged to provide a mean value and standard deviation. Student's t-test was performed for statistical analysis of dose differences between protocols.

All scanning was performed on a 64-MDCT scanner (Discovery CT, 750 HD, GE Healthcare). The phantom was placed in a supine position on the table of the CT scanner at isocenter of the gantry based on laser calipers. A large scan field of view of 50 cm and display FOV of 36 cm was utilized for all protocols. After obtaining initial scout views in the lateral and antero-posterior projections, the phantom was scanned from the lower neck through the pelvis in a cranial to caudal direction. Three separate acquisition protocols were performed with variation in the mA along the z-axis as determined by tube current modulation based on the selected noise index and the scout radiographs.

Two clinical protocols were utilized as follows: (i) sequential protocol with the chest and abdomen/pelvis scanned as sequential acquisitions with noise index of 20 and 15, respectively with a 2.9 second minimum delay between the two anatomic regions, and (ii) single protocol with the chest and abdomen/pelvis scanned as a single acquisition with a NI of 15 with no time delay between anatomic regions.

An experimental protocol utilizing anatomic specific image noise parameters, referred to herein as the Variable Setting Image Noise (ViSIoN) software, was designed as a single acquisition protocol utilizing two different image quality factors along the z-axis course during a single acquisition and was modeled by utilizing varying mA values per helical scan rotation along the z-axis length as can be defined by noise index parameters determined from the scout radiographs from independent anatomic region MDCT acquisitions. The mA values along the z-axis for the prototype ViSIoN software were prescribed by utilizing the mA values from two individual (chest and abdomen/pelvis) anatomic regions for the sequential protocol (see Table 1 below). Anew mA table was created for acquisition of the chest and abdomen/pelvis with the prototype ViSIoN protocol such that the chest and abdomen/pelvis was scanned as a single acquisition with NI of 20 and 15, respectively with no time delay between regions.

TABLE 1
Chest and Abdomen/Pelvis MDCT Protocols

| | | | Minimum | | Total | | | |
|---|---|---|---|---|---|---|---|---|
| | Tube | Noise | Time | Scan | scan | | DLP | Number |
| | current | index | between C | time | time | CTDI | (mGy- | of |
| Protocol | modulation | (NI) | and AP (sec) | (sec) | (sec) | (mGy) | cm) | rotations |
| Sequential | AutomA | 20 | 2.9 | 2.95 c | 9.57 | 4.21 | 762.93 | 14 |
| C&AP | | 15 | | 3.72 a | | 16.25 | | |
| Single | AutomA | 15 | 0 | 6.09 | 6.09 | 12.86 | 824.04 | 13 |
| C&AP | | | | | | | | |
| ViSIoN | AutomA | 20 | 0 | 6.09 | 6.09 | 11.03 | 694.98 | 13 |
| C&AP | | 15 | | | | | | |
| Sequential | Smart mA | 20 | 2.9 | 2.95 c | 9.57 | 3.66 | 823.34 | 14 |
| C&AP | | 15 | | 3.72 a | | 15.72 | | |
| Single | Smart mA | 15 | 0 | 6.09 | 6.09 | 12.30 | 861 | 13 |
| C&AP | | | | | | | | |
| ViSIoN | Smart mA | 20 | 0 | 6.09 | 6.09 | 10.37 | 738.36 | 13 |
| C&AP | | 15 | | | | | | |

All three protocols were evaluated with both Auto mA and Smart mA (GE Healthcare) with a set minimum mA of 100 and a maximum mA of 750. All other scan parameters were kept constant as follows: rotation time of 0.5, pitch of 1:1.375, 64×0.6 mm detector configuration, constant kVp of 120, and scan recon of 5×5 mm (Table 1).

The phantom was scanned three times for each separate protocol to obtain mean organ doses. The minimum and maximum tube current reached for each of the protocols and the mA variation prior to scanning (mA table), total scan time and the dose-length product (DLP) and volume CT dose index (CTDIvol) were recorded from the MDCT scanner for each protocol.

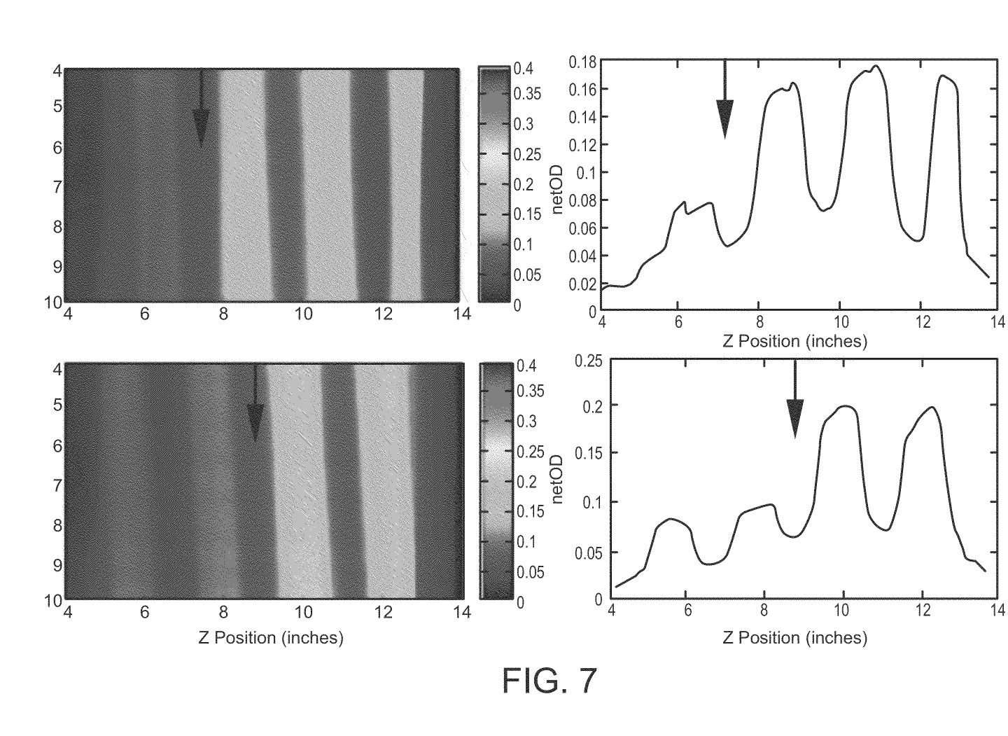

Radiochromic film that has been validated for use in radiation dosimetery was utilized to assess the MDCT beam distribution during the transition from one NI to a different NI for the prototype ViSIoN software. Changes in optical density along the film after exposure to ionizing radiation is linearly proportional to amount of absorbed radiation (reported range of detection is 0.1-20 cGy). A single sheet of radiochromic film (GAFCHROMIC XR-QA2 810, Ashland Inc.; Covington, Ky.) was placed on top of a 32 cm CTDI phantom (FIG. 7) to assess radiation change along the z axis of the phantom and the phantom was scanned utilizing the prototype ViSIoN software with both Auto mA and Smart mA as described above but with a NI of 30 and 15 to assure adequate radiation dose for radiochromic film analysis, pitch of 1.37, rotation time 0.5 total exposure time of 2 seconds. The film was analyzed 24-hours post exposure on an EPSON 10000XL scanner at a resolution of 72 DPI to determine optical density (OD) and netOD was determined to remove any optical scanner artifacts as previously described (11).

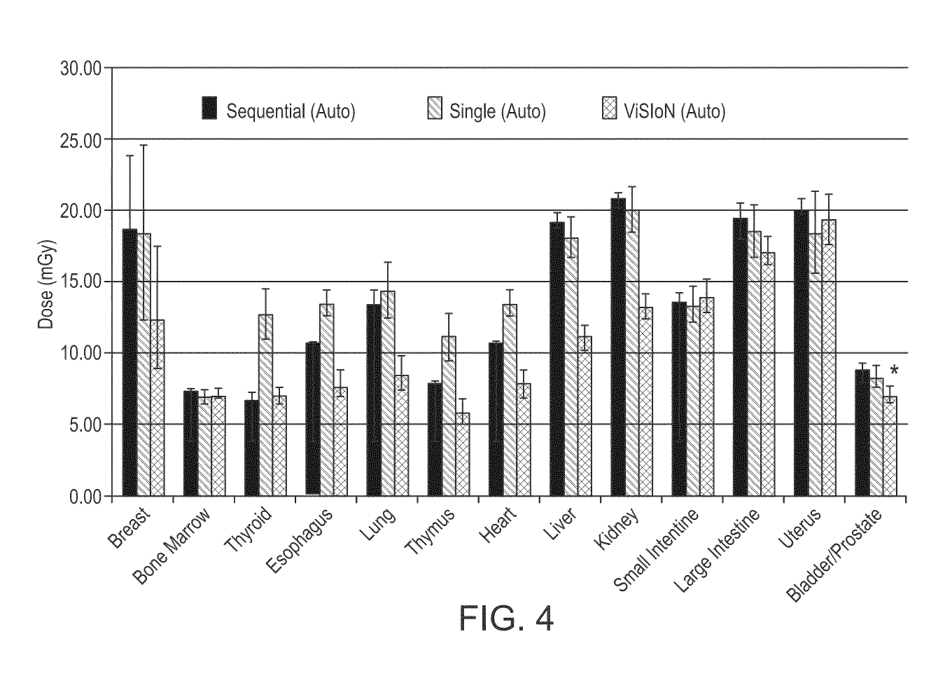

Image quality was quantitatively assessed within the anthropomorphic phantom by measuring the difference in noise in specific anatomic locations in the chest and abdomen at slice levels where there was a transition from one NI to another NI. This comparison was performed between the prototype ViSIoN and the single acquisition and sequential protocols. In the lower chest, two separate 100 mm² circular regions of interest (ROI) were placed in the mediastinum in the midline, as well as in the left lower lung. In the upper abdomen, two separate 100 mm² circular regions of interest (ROI) were placed in the anterior abdominal fat in the midline, as well as in the central abdomen (FIG. 4). The standard deviation of the mean attenuation value (in Hounsfield units) was recorded as the noise level and the results were averaged for each location. A T-test was used to determine statistical significance in noise between the protocols.

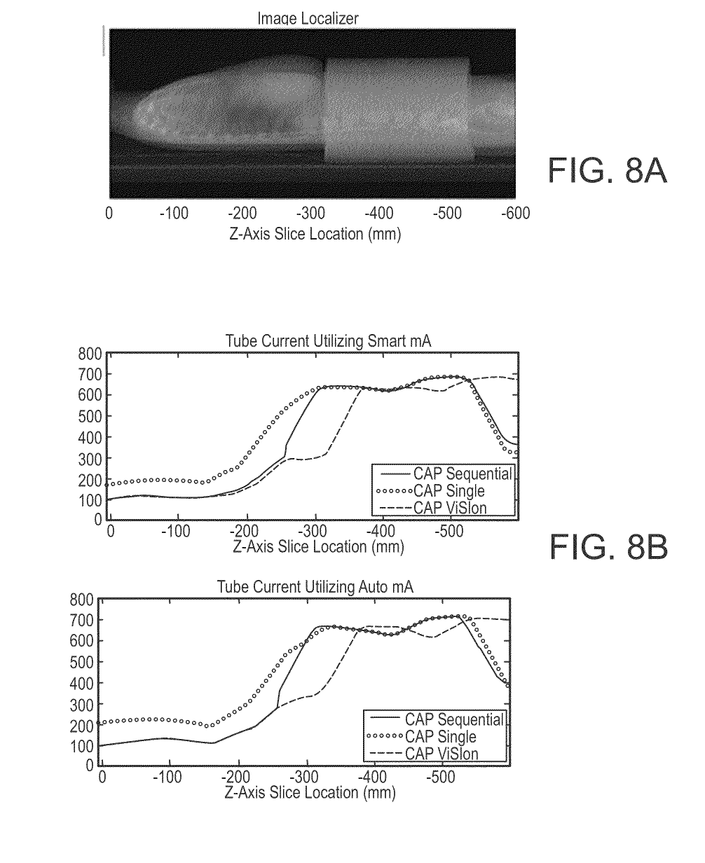

Utilizing a single mA table prescribed from two different NI values resulted in maintaining similar mA values along the length of the chest and the abdomen/pelvis compared to independent image acquisition of the chest and abdomen/pelvis with anatomic specific noise index parameters with both SmartmA and AutomA automatic tube current modulation software. Overall there was a lower mA along the chest for the prototype ViSIoN software as compared to a single acquisition with a single NI (see FIGS. 8A and 8B).

The variation in tube current for all protocols was evaluated as a function of z-axis slice location for both Smart mA and Auto mA (see FIGS. 8A and 8B). For Smart mA, the minimum inflection point for the sequential protocol was at location −250 mm, and for the prototype ViSIoN protocol was at location −320 mm resulting in a shift of 70 mm and at these locations the tube current was 290 mA and 344 mA respectively. For Smart mA, maximum inflection point for the sequential protocol was at location −306 mm, and for the prototype ViSIoN protocol was at location −370 mm resulting in a shift of 64 mm and at these locations the tube current was 631 mA and 627 mA respectively. For Auto mA, minimum inflection point for the sequential protocol was at location −255 mm and for the prototype ViSIoN protocol was at location −320 mm, resulting in a shift of 65 mm and at these locations the tube current was 272 mA and 343 mA respectively. For AutomA, the maximum inflection point for the sequential protocol was at location −301 mm, and for the prototype ViSIoN protocol was at location −370 mm, resulting in a shift of 69 mm and at these locations the tube current was 618 mA and 620 mA, respectively. The inflection point for change in mA was constant for both the prototype ViSIoN acquisition utilizing smart mA and Auto mA. For both Smart mA and Auto mA, there was a shift in the change in mA along the course of the z-axis for the prototype ViSIoN software as compared to the independent acquisitions for approximately 64-69 mm (1.5 gantry rotations) resulting in a lower mA for the upper abdomen for the prototype ViSIoN software as compared to the separate image acquisitions.

The highest organ doses were for the single acquisition protocol utilizing a single NI along the entire z-axis. On average the prototype ViSIoN protocol demonstrated the greatest decrease in absorbed organ dose compared to the single acquisition protocol and this was most notable in the chest and upper abdomen; the greatest dose reduction with the prototype ViSIoN software was in the liver (43% lower), lung (36% lower), kidney (36% lower), esophagus (22% lower), thymus (47% lower) and breast (33% lower) all of which were statistically significant (p=<0.05) (Table 2).

TABLE 2
Absorbed organ dose with standard deviation for chest and abdomen/pelvis sequential, single
and prototype ViSIoN protocols utilizing AutomA and Smart mA tube current modulation.

| | Sequential | Sequential | Single | Single | | ViSIoN |
|---|---|---|---|---|---|---|
| | C&AP | C&AP | CAP | C&AP | ViSIoN | C&AP |
| | NI 20&15 | NI 20&15 | NI 15 | NI15 | C&AP | NI15 |
| | (Auto) | (Smart) | (Auto) | (Smart) | NI15 (Auto) | (Smart) |
| Organ | Dose (mGy) | Dose (mGy) | Dose (mGy) | Dose (mGy) | Dose (mGy) | Dose (mGy) |
| Breast | 18.78 ± 5.22 | 22.60 ± 1.67 | 18.54 ± 6.07 | 24.33 ± 3.17 | 12.48 ± 3.55 | 9.87 ± 3.44 |
| Bone Marrow | 7.36 ± 0.11 | 6.45 ± 0.24 | 7.02 ± 0.45 | 6.40 ± 0.23 | 7.20 ± 0.34 | 6.84 ± 0.61 |
| Thyroid | 6.78 ± 0.55 | 7.28 ± 0.55 | 12.83 ± 1.82 | 8.99 ± 1.63 | 7.11 ± 0.55 | 5.92 ± 1.43 |
| Esophagus | 10.75 ± 0.00 | 10.57 ± 1.43 | 13.60 ± 0.90 | 13.33 ± 1.05 | 7.93 ± 0.92 | 8.50 ± 1.96 |
| Lung | 13.58 ± 1.00 | 11.36 ± 1.02 | 14.51 ± 1.97 | 14.06 ± 2.05 | 8.70 ± 1.17 | 7.40 ± 1.11 |
| Thymus | 7.83 ± 0.29 | 6.67 ± 0.76 | 11.25 ± 1.71 | 9.50 ± 2.52 | 6.00 ± 0.82 | 5.25 ± 0.96 |
| Heart | 10.75 ± 0.00 | 10.57 ± 1.43 | 13.60 ± 0.90 | 13.33 ± 1.05 | 7.93 ± 0.92 | 8.50 ± 1.96 |
| Liver | 19.50 ± 0.40 | 19.68 ± 0.81 | 18.28 ± 1.39 | 18.60 ± 0.20 | 11.19 ± 0.95 | 10.42 ± 0.80 |
| Kidney | 20.92 ± 0.42 | 20.67 ± 0.50 | 20.20 ± 1.65 | 20.10 ± 0.57 | 13.40 ± 0.92 | 12.70 ± 0.50 |
| Small Intestine | 13.82 ± 0.49 | 13.23 ± 0.68 | 13.60 ± 1.21 | 12.38 ± 0.38 | 14.08 ± 1.10 | 13.33 ± 0.85 |
| Large Intestine | 19.67 ± 0.90 | 18.33 ± 1.17 | 18.70 ± 1.86 | 18.20 ± 0.65 | 17.30 ± 1.00 | 17.10 ± 1.44 |
| Uterus | 19.98 ± 0.77 | 16.02 ± 0.63 | 18.58 ± 2.86 | 16.28 ± 1.27 | 19.45 ± 1.84 | 19.63 ± 2.35 |
| Bladder/Prostate | 9.07 ± 0.29 | 7.95 ± 0.09 | 8.39 ± 0.73 | 7.30 ± 0.32 | 12.50 ± 0.91 | 10.32 ± 0.88 |
| Rescaled | n/a | n/a | n/a | n/a | 7.10 ± 0.52 | 6.09 ± 0.52 |
| Bladder/Prostate | | | | | | |

Auto = automA
Smart = Smart mA
* breast denotes average of left and right breast doses and bone marrow denotes average of bone marrow in the mandible, thoracic and lumbar spine.

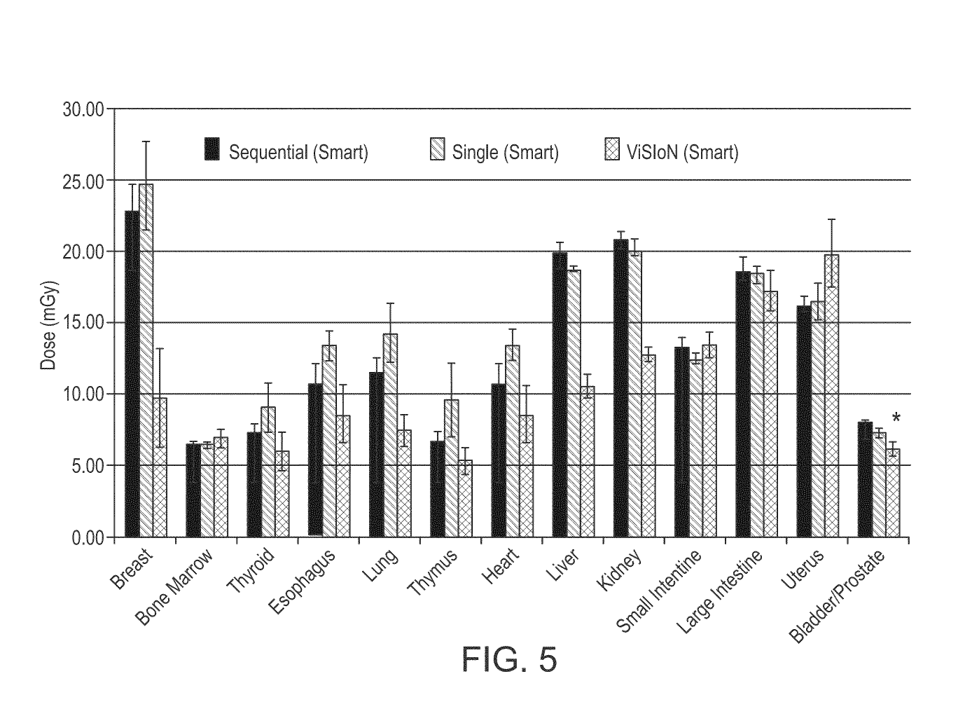

There was a recorded increase in the dose to both the bladder/prostate (38% increase) and thyroid (4.7% increase) for the prototype ViSIoN software (Table 2, 3; FIGS. 4, 5, 8A, and 8B). The bladder/prostate dose increase was due to prototype ViSIoN software parameters and resultant shift in mA caudally as compared to a sequential mode of scanning so that at the same location in the pelvis the mA was different due to shift in inflection point. As the tube current (mA) is directly proportional to radiation dose, the dose to the bladder/prostate was rescaled so that the mA in the prototype ViSIoN protocol can match the mA in the respective slice of the sequential acquisition as one would expect with no shift in the inflection point. This was done by multiplying the bladder/prostate dose in the prototype ViSIoN scan by the ratio of the Sequential mA to the prototype ViSIoN mA:

$${Dose}_{rescaled} = {Dose}_{ViSIoN} ( \frac{{mA}_{Sequential}}{{mA}_{ViSIoN}} )$$

In this equation, Dose_{rescaled} is the rescaled dose to the bladder/prostate, Dose_{ViSIoN}

is the original dose to the bladder/prostate in the prototype ViSIoN scan, and mA_{Sequential} and mA_{ViSIoN} are the tube currents at the slice where the bladder/prostate dose measurement was taken in the Sequential and prototype ViSIoN scans, respectively. Using this information, the rescaled bladder/prostate dose for the Prototype ViSIoN scan was 7.10±0.52 mGy and 6.09±0.52 mGy for the scans utilizing auto mA and smart mA, respectively (Table 4).

TABLE 3
Quantitative noise measurements (HU) for Sequential
acquisition and ViSIoN protocols.

| | Noise | Noise | | Noise |
|---|---|---|---|---|
| | Center | Anterior | Noise | Center |
| Protocol | Abdomen | abdomen | Left lung | (heart) |
| C& AP sequential | 23.5 ± 19 | −65.1 ± 14 | −776 ± 18 | 30 ± 25 |
| ViSIoN | 22.8 ± 19 | −63.8 ± 13 | −778.5 ± 16 | 29.5 ± 22 |
| p-values | 0.98 | 0.95 | 0.94 | 1.0 |

p-value of <0.05 is considered statistically significant.

TABLE 4
Measured organ dose values for the slice where the
bladder/prostate dose measurement was taken for both
the Sequential and prototype ViSIoN scans.

Bladder/Prostate Dose (mGy)

| | Auto | Smart |
|---|---|---|
| C& AP | 9.07 (±0.29) | 7.95 (±0.09) |
| Sequential | | |
| C&AP | 12.5 (±0.91) | 10.3 (±0.88) |
| ViSIoN | | |
| C& AP ViSIoN | 7.10 ± 0.52 | 6.09 ± 0.52 |
| (Rescaled) | | |

In addition to organ dose savings, total exposure was decreased as noted by the reduction in DLP for the prototype ViSIoN software as compared to the two other protocols (Table 1). DLP was lowered by average of 10.6% for the prototype ViSIoN protocol as compared to the dual NI sequential protocol, 11.5% and 9.8% for Smart mA and AutomA respectively. DLP was lowered by average of 17.6% for the prototype ViSIoN protocol as compared to the sequential single NI protocol, 16.6% and 18.6% for SmartmA and AutomA respectively.

Overall scan time was lowest during the single acquisition chest and abdomen/pelvis scanning protocol and the prototype ViSIoN protocol (6.09 s) compared to the sequential acquisition for the chest and abdomen/pelvis protocol (9.57 s) (Table 2) which resulted in a savings of 3.48 seconds.

The radiochromic film analysis demonstrates distinct radiation bands with alternating high/low magnitudes along the z-axis resulting from differences in the radiation flux incident on the film as the tube rotated around the phantom. The exposure bands are separated at fixed axial distances which reflect the pitch of the scan. The dose in the central part of the direct beam was found to change by a factor of 2.5 within a single tube rotation from a value of about 1 cGy at axial position 6.5″ to 2.5 cGy at a position of 8.5″ (FIG. 7). Because dose scales linearly with mA, and mA is varied according to the desired NI, the boundary of the NI change is able to be identified. There were no intermediate radiation beam doses recorded to suggest that the prototype ViSIoN software protocol resulted in abrupt (ie. faster than a single tube rotation).

Variation in noise between the regions of the phantom with the mA change in the prototype ViSIoN software protocol compared to the sequential protocol demonstrated no significant change in image noise between the protocols in the regions of the upper abdomen and lower chest, at slice locations where N1 change was known to occur (Table 2).

The prototype ViSIoN software provides a novel method to optimize image quality and minimize radiation exposure over an expanded z-axis for MDCT imaging protocols. This new method of image acquisition is accomplished by allowing for selective choice of anatomic specific noise indices (in this example based on mA though other parameters may be changed in similar accordance) across the chosen z-axis of image acquisition. This study was designed to verify the protocol design related to image quality and measured absorbed organ dose. Our data demonstrate that the proposed software can reduce total radiation exposure as noted by reduced DLP values as well as reduced organ absorbed dose to radiosensitive organs of the chest while consecutively imaging the abdomen and pelvis without incurring a time penalty, both of which are drawbacks of current combined chest and abdomen/pelvis imaging protocols. Dose savings to the chest were substantial at approximately 33-36% with no significant change in organ dose to the abdomen and one can foresee a higher realized dose savings in clinical practice with the prototype ViSIoN protocol depending on the NI levels utilized. The recorded higher doses to the organs in pelvis in our study can be alleviated with shift in inflection point and modification of the mA table to remove any shift in mA change at point of switch in NI.

During the creation of the mA table for the prototype ViSIoN protocol the mA values prescribed from the two individual anatomical regions were combined into one data set, without accounting for overlap region at the boundaries of the two anatomical regions. Since the prototype ViSIoN software takes input mA values per rotation, this combination led to combining mA values from both regions, one from the last rotation of first anatomical region and one from the first rotation of the second anatomical region with a resultant shift in the mA and change in inflection point for transition from one NI value to a different NI value. Other embodiments of this prototype-software can exploit this transition point to either increase or decrease the time to transition or shift the transition point may optimize the mA switch during the transition from the chest to the abdomen/pelvis.

Maintenance of image quality for the prototype ViSIoN protocol as noted by quantitative measurement of change in noise is likely due to rapid shift in mA confirmed by the radiochromic film analysis with absence of intermediary beams during the NI switch. The added benefit of a time saving of 3.46 seconds for the prototype ViSIoN software as compared to a sequential acquisition may lead to reduction in intravenous contrast volumes and motion artifacts and improvement in image quality in the clinical setting.

This study describes a critical first step to modifying clinical CT scanning protocols to utilization of anatomic and pathology specific parameters during single image acquisition over a long z-axis coverage. There are many applications for this software, including malignancy staging or follow-up, trauma, and thoracoabdominal angiographic imaging. While our study specifically focused on chest and abdomen/pelvis CT imaging, combinations of various indications and anatomic areas also can be envisioned for which single acquisition is important; such as combined neck and thoracic imaging and combined chest and abdomen/pelvis and lower extremity imaging, for soft tissue and vascular imaging indications.

In conclusion, dual noise level settings during single helical acquisition of chest and abdomen/pelvis MDCT imaging are technically feasible. When compared to both single chest and abdomen/pelvis acquisition with one noise setting and sequential chest and abdomen/pelvis scan acquisition with specified anatomically adjusted noise settings, this technique was shown to reduce radiation dose and scan time without loss in image quality. Further, clinically based investigation on this MDCT scanning technology may explore applications in other anatomic parts of the body, ideal clinical strategies for implementation, further software modifications in transitioning the noise index switching (and other CT) parameters and use for axial acquisition as well as helical acquisitions.

While current work demonstrates utility for helical image acquisition, use of for example, a GE 64 MDCT scanner and change in noise index only, this work can also be applied for axial image acquisition, 16-320 or higher MDCT CT, and for other CT parameters, ie., rotation time, pitch, kVp, and the like.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. Anon-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flow chart illustrations and/or block diagrams of methods, apparatus (systems), and computer program products according to embodiments of the subject matter. It will be understood that each block of the flow chart illustrations and/or block diagrams, and combinations of blocks in the flow chart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flow chart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flow chart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flow chart and/or block diagram block or blocks.

The flow chart and block diagrams in the Figures illustrate the architecture, functionality, and operation of possible implementations of systems, methods, and computer program products according to various embodiments of the present subject matter. In this regard, each block in the flow chart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flow chart illustration, and combinations of blocks in the block diagrams and/or flow chart illustration, can be implemented by special purpose hardware-based systems that perform the specified functions or acts or carry out combinations of special purpose hardware and computer instructions.

Any patents or publications mentioned in this specification are indicative of the levels of those skilled in the art to which the present subject matter pertains. These patents and publications are herein incorporated by reference to the same extent as if each individual publication was specifically and individually indicated to be incorporated by reference.

One skilled in the art will readily appreciate that the present subject matter is well adapted to carry out the objects and obtain the ends and advantages mentioned, as well as those inherent therein. The present examples along with the methods described herein are presently representative of various embodiments, are exemplary, and are not intended as limitations on the scope of the present subject matter. Changes therein and other uses will occur to those skilled in the art which are encompassed within the spirit of the present subject matter as defined by the scope of the claims.

What is claimed is:

1. A method for computed tomography (CT) imaging, the method comprising:
using a CT system to capture CT image data of an object in a single acquisition;
adjusting one or more image quality factors of the CT system during capture of the CT image data of the object; and
processing the CT image data for presentation based on the one or more image capture settings.

2. The method of claim 1, wherein using the CT system comprises:
during the single acquisition:
capturing a first set of CT image data with the CT system set to a first image quality factor or CT capture setting; and
capturing a second set of CT image data with the CT system set to a second image quality factor or CT capture setting, wherein the first image quality factor or CT capture setting image capture setting is different than the second image quality factor or CT capture setting.

3. The method of claim 2, wherein the first and second image quality factor will affect the CT capture settings during the CT examination and these CT capture settings may include one of tube current, kilovoltage peak, field of view, pitch, and rotation time.

4. The method of claim 1, wherein the object is a human, and
wherein using the CT system comprises:
during the single acquisition:
capturing a first set of CT image data of a first portion of the human with the CT system set to a first image quality factor or CT parameter setting; and
capturing a second set of CT image data of a second portion of the human with the CT system set to a second image quality setting or CT parameter setting, wherein the first image quality setting or CT parameter setting is different than the second image quality factor of CT parameter setting.

5. The method of claim 4, wherein the first portion is an upper portion of the human, and the second portion is a lower portion of the human.

6. The method of claim 1, further comprising assessing the processed CT image data based on image quality.

7. The method of claim 1, further comprising determining radiation dosage levels at different portions of the object subsequent to use of the CT system.

8. The method of claim 1, wherein using the CT system to capture CT image data comprises generating X-rays and directing the X-rays towards the object.

9. The method of claim 8, wherein using the CT system to capture CT image data comprises detecting the X-rays after interaction of the X-rays with the object.

10. The method of claim 1, further comprising presenting an image based on the processed CT image data.

11. The method of claim 9, wherein presenting the image comprises displaying the image.

12. A computed tomography (CT) system comprising:
an X-ray source and detector configured to capture CT image data of an object in a single acquisition; and
at least one processor and memory configured to:
adjust one or more image capture settings of the CT system during capture of the CT image data of the object; and
process the CT image data for presentation based on the one or more image quality factors or image capture settings.

13. The CT system of claim 12, wherein the X-ray source and detector are configured to:
capture, during the single acquisition, a first set of CT image data with the CT system set to a first image quality factor or image capture setting; and
capture, during the single acquisition, a second set of CT image data with the CT system set to a second image quality factor or image capture setting, wherein the first image quality factor or image capture setting is different than the second image capture setting.

14. The CT system of claim 13, wherein the first and second capture settings include one of current, kilovoltage peak, field of view, and rotation time.

15. The CT system of claim 12, wherein the object is a human, and
wherein the X-ray source and detector are configured to:
capture, during the single acquisition, a first set of CT image data of a first portion of the human with the CT system set to a first image quality factor or image capture setting; and
capture, during the single acquisition, a second set of CT image data of a second portion of the human with the CT system set to a second image quality factor or image capture setting, wherein the first image quality factor or image capture setting is different than the second image quality factor or image capture setting.

16. The CT system of claim 15, wherein the first portion is an upper portion of the human, and the second portion is a lower portion of the human.

17. The CT system of claim 12, wherein the at least one processor and memory are configured to assess the processed CT image data based on image quality.

18. The CT system of claim 12, wherein the at least one processor and memory are configured to determine radiation dosage levels at different portions of the object subsequent to use of the CT system.

19. The CT system of claim 12, wherein the X-ray source and detector are configured to generate X-rays and direct the X-rays towards the object.

20. The CT system of claim 19, wherein the X-ray source and detector are configured to detect the X-rays after interaction of the X-rays with the object.
